(19) United States
(12) Patent Application Publication
(10) Pub. No.: US 2011/0106266 A1
(43) Pub. Date: May 5, 2011

(54) SCAPULAR COMPONENT OF A SHOULDER JOINT PROSTHESIS

(75) Inventors: Hans-Kaspar Schwyzer, Zurich (CH); Marc A. Riner, Aristau (CH); Ernst Kehrli, Rotkreuz (CH)

(73) Assignee: SMITH & NEPHEW ORTHOPAEDICS AG, Memphis, TN (US)

(21) Appl. No.: 12/988,044

(22) PCT Filed: Apr. 14, 2009
(86) PCT No.: PCT/EP09/02734

§ 371 Date: Jan. 21, 2011

(30) Foreign Application Priority Data
Apr. 28, 2008 (DE) 10 2008 021 110.9

(51) Int. Cl. A61F 2/40

(52) U.S. Cl. 623/19.11

(57) ABSTRACT

Scapular component of a shoulder joint prosthesis, having a base plate (11) anchorable to the scapula and, affixable to the base plate, a joint surface member (12) which defines either an anatomical (concave, 13) joint surface or a reverse (convex, 33) joint surface. On the rear side (14) of the base plate (11), which faces the scapula in the implanted state, there is arranged at least one anchoring pin (15, 16, 17) extending obliquely with respect to the base plate (11) in approximately the anterolateral direction.

The present invention relates to a scapular component of a shoulder joint prosthesis, having a base plate anchorable to the scapula and, affixable to the base plate, a joint surface member which defines either an anatomical (concave) joint surface or a reverse (convex) joint surface.

Such scapular components are generally known. In this respect reference is made only by way of example to EP 1 520 561 A1.

In joint surface replacement at the scapula-side glenohumeral joint, the main difficulty lies primarily in good exposure of the original joint socket in the case of the deltopectoral approach which is usually practised. If the humerus is provided with a surface replacement prosthesis, that approach is additionally rendered difficult because the anatomical humerus head is not resectioned as in the case of a stem prosthesis, but only the surface thereof is prepared. This is the main reason why surface replacement prostheses are implemented almost exclusively in the form of hemiarthroplasties.

The revision of anatomical shoulder systems to reverse shoulder systems in the case of conventional glenoid systems necessitates explantation of the existing glenoid implant, which results in a high degree of traumatisation of the glenoid bone.

If replacement of the joint surface of a glenoid is necessary, then for the reasons mentioned above a stem prosthesis must be selected, although on the humerus side the indication would be for a surface replacement prosthesis. This results in a comparatively high loss of bone at the humerus, which can have an adverse effect on any later revisions in respect of anchoring the revision implant.

Starting from the quoted prior art, the present invention is based on the problem of providing a glenoid system which allows implantation from the anterolateral direction, thus facilitating the approach to the joint surface of the scapula-side glenohumeral joint. At the same time it is to be possible to use, as desired, a joint surface member having an anatomical, i.e. concave, bearing surface or a joint surface member having a reverse, i.e. convex, bearing surface, so that in the event of revision it is also possible, if necessary, to change from an anatomical system to an inverse system without its being necessary for the base plate already anchored in the bone to be explanted.

That problem is solved according to the invention by the characterizing features of claim 1, with further details and preferred embodiments being described in the subsidiary claims.

A characteristic of the invention is, therefore, inter alia that at least one anchoring pin is arranged on the rear side of the base plate, which faces the scapula in the implanted state, the anchoring pin extending obliquely with respect to the base plate in approximately the anterolateral direction.

Preferably, on the rear side of the base plate there are provided two, especially three, anchoring pins extending obliquely with respect to the base plate. The anchoring pins extend within a predetermined angle range, especially within an angle range of from 30° to 70° relative to the base plate. Accordingly, implantation from the anterolateral direction is possible. The base plate has the basic shape of an approximately circular, but especially oval or pear-shaped, disc. The afore-mentioned anchoring pins can extend either in parallel or, at least in part, diverging from one another, but always in approximately the anterolateral direction. That variant allows a cementless surgical technique. The base plate itself consists preferably of a Ti alloy, which for the purpose of improved osteointegration can be provided with a porous titanium/hydroxyapatite coating or like bioactive coating. The joint surface member already mentioned is affixed to the front side, that is to say to the side remote from the scapula in the implanted state, the joint surface member preferably consisting of polyethylene, or PE, in the case of an anatomical procedure. In the case of a reverse procedure, the joint surface member is made of ceramics or implant steel. Because the materials for such joint members are known per se, a more detailed description is unnecessary here.

In a preferred embodiment, the PE insert, which in the case of an anatomical procedure defines the actual joint surface, is mounted by way of a mechanism which snaps together with the base plate either in situ or extracorporeally.

Preferably, there is also provided a so-called primary anchoring by means of at least one bone screw, preferably two bone screws, which pass through a corresponding bore in the base plate. Inside that bore the bone screws should extend in an angular region between base plate and anchoring pin, especially in the region of the angle bisecting line between base plate and anchoring pin. In the case of more than one bone screw, the bone screws extend either in parallel or diverging from one another in a range of from 0° to 45°.

In the event of revision, the mentioned PE insert is demounted using an extraction instrument and replaced by a hemispherical joint surface member or a so-called glenosphere. The glenosphere can be positioned centrally or eccentrically relative to the geometric centre axis of the base plate. In structural terms the anchoring between glenosphere and base plate is such that the glenosphere is anchorable on the base plate from the anterolateral direction. For that purpose there are preferably formed on the front side of the base plate at least one, especially two, guide grooves or guide ribs extending approximately anteroposteriorly in the implanted state, which grooves or ribs correspond to complementary guide elements on the underside of the glenosphere. The guide grooves can be of dovetail-like construction in cross-section. Preferably, the guide grooves so constructed taper conically in the direction from anterior to posterior, so that on being fixed on the base plate the glenosphere cannot be displaced beyond the guide grooves in the anterior direction. The fixing of the glenosphere on the base plate is then effected by means of a securing screw which corresponds to an internal thread inside an anchoring pin arranged approximately centrally on the underside of the base plate.

The surgical technique provides for the preparation of an implant bed by means of a rasp instrument which is guided free-hand and which is driven by a drill or the like.

By means of a drilling jig, oblique anchoring holes corresponding to the afore-mentioned anchoring pins on the underside of the base plate are then drilled in the bone. Using a positioning instrument, the base plate is introduced into the prepared cavity. Fixation is then effected by means of the mentioned bone screws which are of self-drilling and self-tapping construction.

The rear side of the base plate can either be of flat or flat-spherical, that is to say slightly domed, construction.

It has already been pointed out that the anchoring screws can be positioned so that they diverge from one another, more specifically in a range between 0° and 45°.

It can be provided that the bone screws are held on the base plate either unidirectionally or polydirectionally. In both cases the necessary angular stability must, of course, be ensured but this involves technology known per se.

It is also conceivable to use conventional bone screws. Alternatively, the use of non-fixed-angle, self-drilling and self-tapping bone screws is also conceivable.

The anterolateral approach allows treatment of the scapula-side glenohumeral joint with a glenoid implant in combination with a humerus-side surface replacement prosthesis. In addition, it is possible to change from an anatomical system to a reverse system at a later date. Both the primary procedure and the revision procedure are less invasive than with conventional implant systems. The patient is thus subjected to less stress and rehabilitation takes place more quickly.

A preferred embodiment of a scapular component according to the invention having an anatomical or, alternatively, a reverse joint surface member will be described below with reference to the accompanying drawing, wherein:

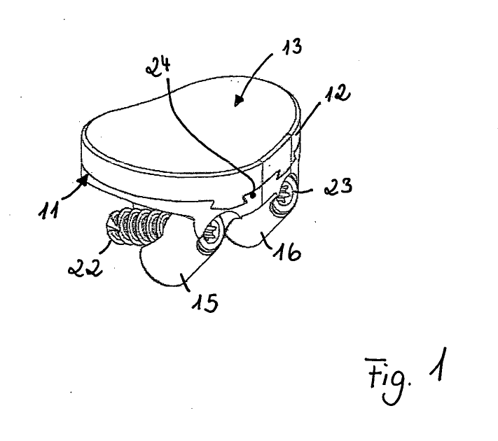

FIG. 1 shows a glenoid having an anatomical joint surface member for an antero-lateral approach in a perspective view obliquely from above;

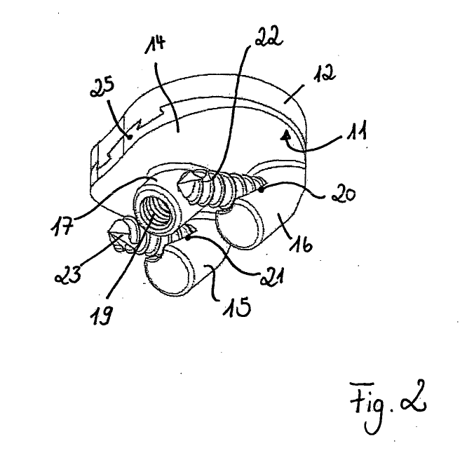

FIG. 2 shows the glenoid according to FIG. 1 in a perspective view obliquely from below;

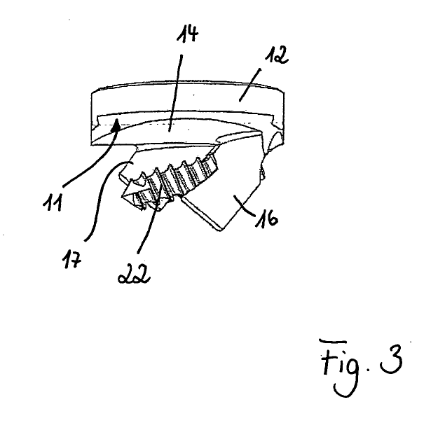

FIG. 3 shows the glenoid according to FIGS. 1 and 2 viewed from the inferior direction;

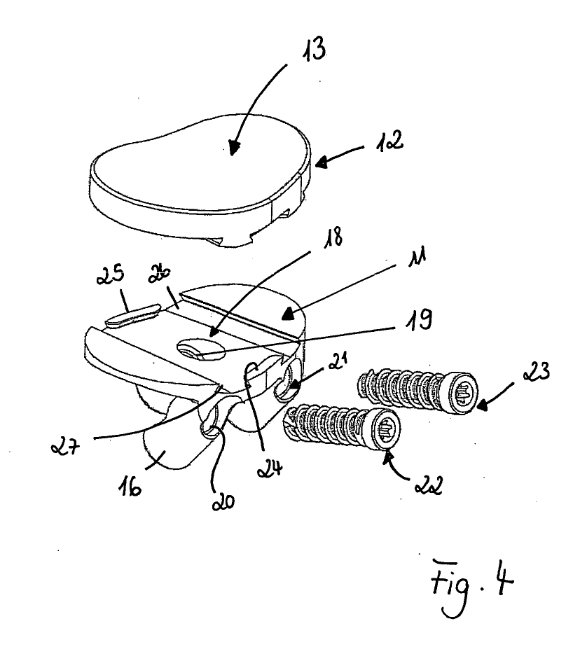

FIG. 4 shows the glenoid according to FIGS. 1 to 3 in an exploded view (perspective view);

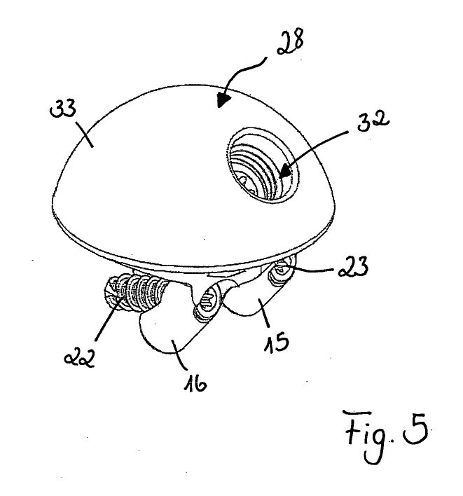

FIG. 5 shows a glenosphere positioned centrally on a base plate for an antero-lateral approach in a perspective view obliquely from above;

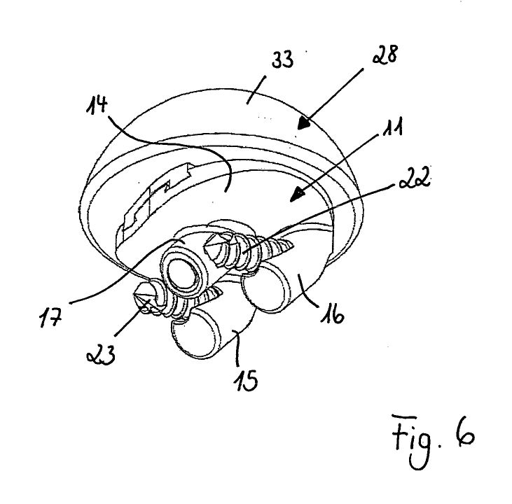

FIG. 6 shows the glenosphere according to FIG. 5 in a perspective view obliquely from below;

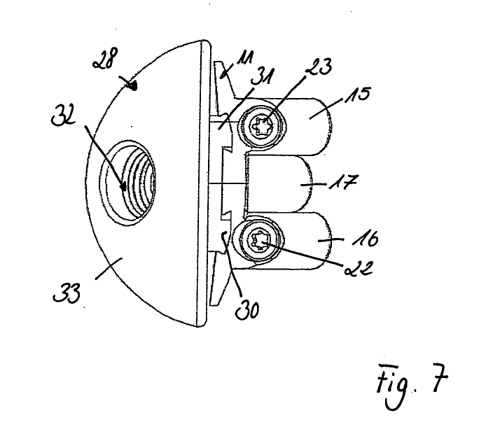

FIG. 7 shows the glenosphere according to FIGS. 5 and 6 in a view from the anterior direction;

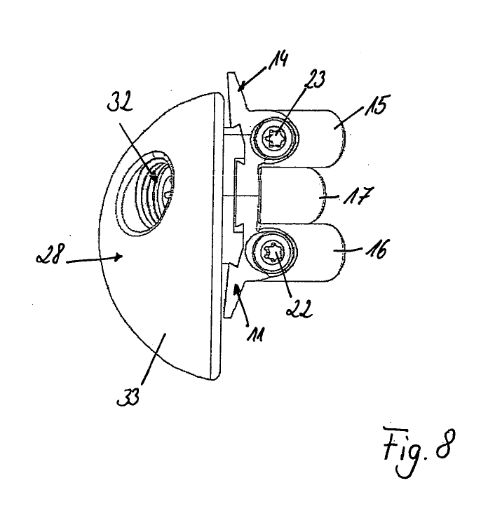

FIG. 8 shows a glenosphere anchored eccentrically relative to the base plate in a view from the anterior direction;

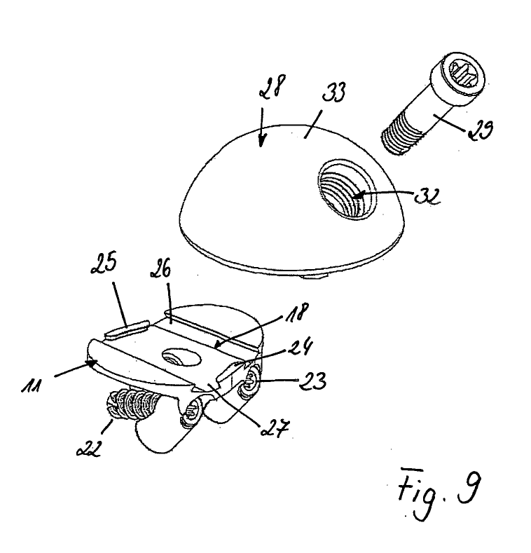

FIG. 9 shows the glenosphere according to FIGS. 5 to 7 in an exploded view obliquely from above;

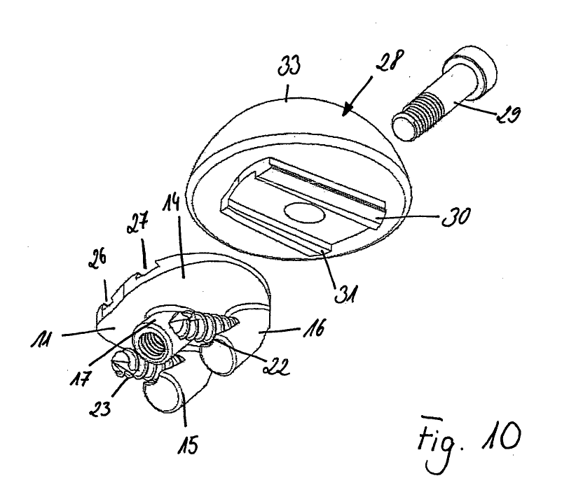

FIG. 10 shows the glenosphere according to FIGS. 5 to 7 in an exploded view obliquely from below;

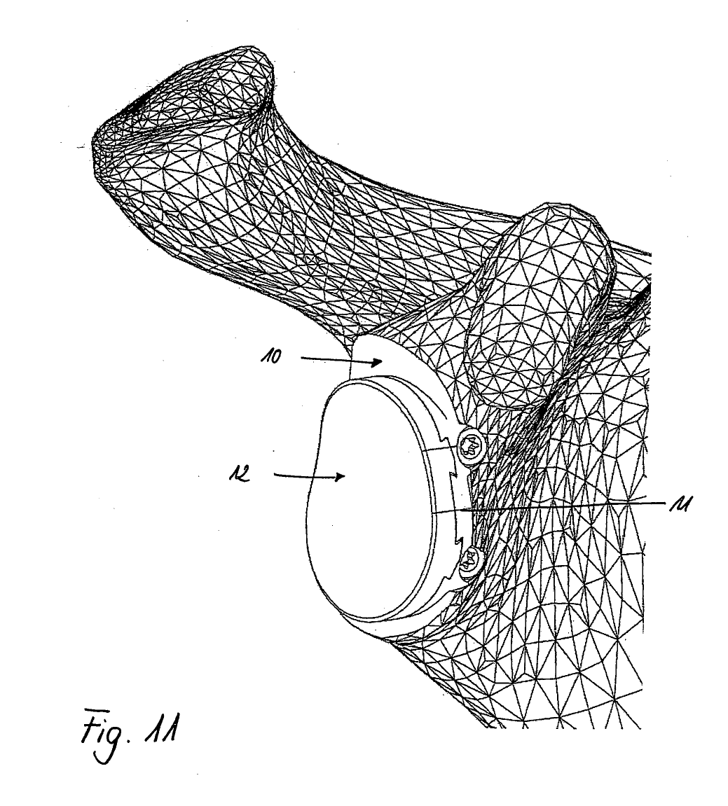

FIG. 11 shows a glenoid corresponding to FIGS. 1 to 4 with scapula;

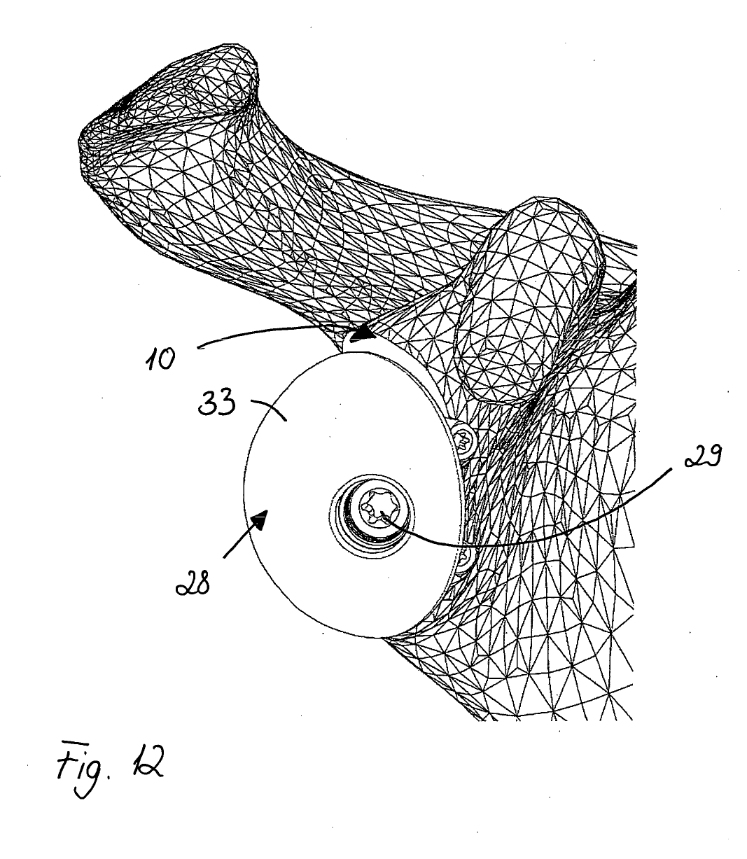

FIG. 12 shows a glenosphere according to FIGS. 5 to 7 centrally with scapula; and

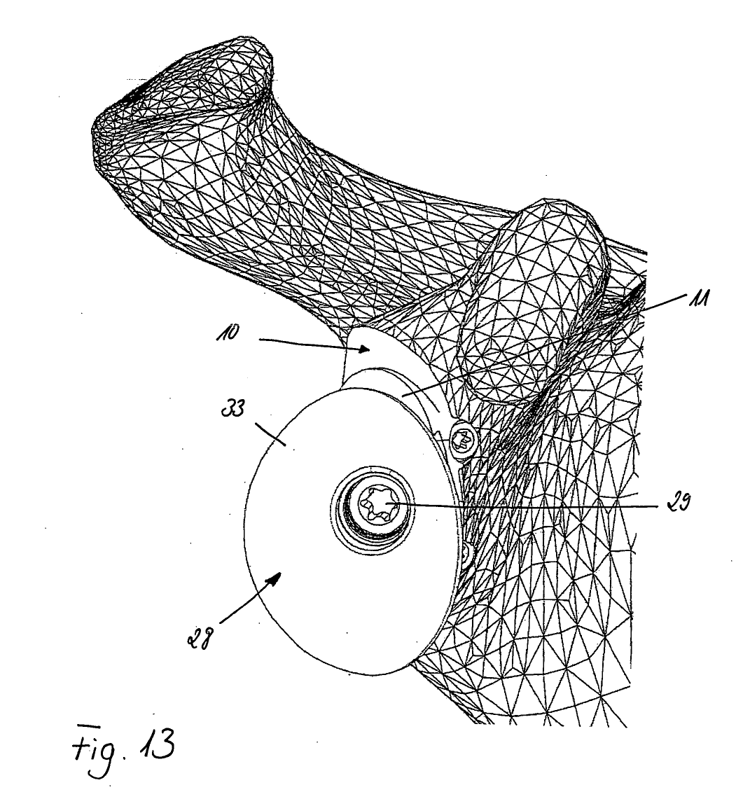

FIG. 13 shows a glenosphere corresponding to FIG. 8 eccentrically with scapula.

FIGS. 1 to 4 show a scapular component of a shoulder joint prosthesis, more specifically a so-called glenoid having a base plate 11 anchorable to the scapula 10 (see FIGS. 11 to 13) and, affixable to the base plate, a joint surface member which defines an anatomical, i.e. concave, joint surface 13. On the rear side 14 of the base plate 11 facing the scapula 10 in the implanted state, which rear side can be of either flat or, as here, slightly domed construction, there are arranged three anchoring pins 15, 16 and 17 each extending obliquely with respect to the base plate 11 in approximately the anterolateral direction. Specifically, all three anchoring pins extend parallel to one another within a predetermined angle range, especially an angle range of from 30° to 70° relative to the base plate, in this case approximately 45° relative to the base plate 11.

Two anchoring pins, namely the two anchoring pins 15 and 16, are arranged at the edge on the rear side of the base plate 11, while the third anchoring pin 17 adjoins the rear side 14 of the base plate 11 approximately centrally. That approximately centrally arranged anchoring pin 17 bounds a threaded bore 19 which is accessible from the front side 18 of the base plate 11 (see FIG. 4), to which the joint surface member, here preferably PE insert 12, is attachable. Into that threaded bore 19 there is screwable a securing screw by means of which a joint surface member is fixable on the base plate. This is described in greater detail with reference to FIGS. 5 to 10.

In the embodiment shown, for the primary anchoring of the base plate 11 the latter has two bores 20, 21 for receiving a bone screw 22, 23, respectively, in such a way that the bone screws extend in the angular region between base plate and anchoring pin 15, 16, especially in the region of the angle bisecting line between base plate 11 and the afore-mentioned anchoring pins 15, 16. In that respect the fixing of the bone screws 22, 23 is possible or achieved also from the anterolateral direction.

The bone screws 22, 23 can be held inside the associated bores 20, 21 in the base plate 11 either unidirectionally or polydirectionally, this being technology known per se which need not be described in greater detail here. It has also already been pointed out that in the case of two or more bone screws 22, 23 the latter extend either in parallel, as here, or alternatively diverging from one another, the divergence in the latter case being in the range of from 0° to 45°.

In the present case the bone screws 22, 23, which are each of self-tapping construction, extend at an angle of about from 20° to 25° relative to the base plate 11.

In the embodiment shown, the joint surface member, preferably PE insert 12, is lockable onto the front side 18 of the base plate 11. For that purpose, the front side 18 of the base plate 11 has two locking projections 24, 25 (see especially FIG. 4 in this connection) which are arranged at the edge and which correspond to complementary locking openings in the glenoid or PE insert 12. The arrangement of locking projection and locking opening can, of course, also be reversed.

Those locking projections can be brought into a disengaged position by means of an extraction instrument so that in the event of a revision the PE insert or the glenoid 12 can be removed.

Because there are also provided on the front side 18 of the base plate 11 dovetail guide means 26, 27 which taper conically from anterior to posterior for anchoring a so-called glenosphere, which will be described in greater detail hereinbelow, the glenoid 12 has on its underside corresponding or complementary projections which fill the afore-mentioned dovetail guide means. The attachment of the glenoid 12, however, should especially take place by means of the afore-mentioned locking mechanism. If that locking mechanism were to be omitted, the glenoid would have to be fixed by means of a securing screw, as described below with reference to the fixing of a glenosphere on the base plate. Preference is given to attachment by means of a locking mechanism in combination with a dovetail guide means; it is then possible to dispense with a securing screw.

As already mentioned, in the event of revision the glenoid 12 can be replaced by a reverse joint surface member or a so-called glenosphere 28. Such a joint surface member or such a glenosphere is fixed to the base plate 11 by means of a securing screw 29 (see FIGS. 9, 10 and 12, 13) passing through the joint surface. The construction of the base plate 11 is as described with reference to FIGS. 1 to 4. The securing screw 29 is screwable into the threaded bore 19 formed in the approximately centrally arranged anchoring pin, likewise from the anterolateral direction. As already mentioned, on the front side 18 of the base plate 11 there are formed two guide grooves which extend approximately anteroposteriorly in the implanted state and which are in the form of dovetail guide means 26, 27 that taper conically in the direction from anterior to posterior. Those dovetail guide means 26, 27 correspond to complementary guide elements, namely guide rails 30, 31 on the underside of the respective associated glenosphere 28.

The glenosphere can be positioned relative to the base plate 11 either centrally as in FIGS. 5 to 7 or eccentrically as in FIG. 8.

It should also be pointed out that for the purpose of fixing the glenosphere 28 on the base plate 11 the securing screw 29 passes through a bore 32 in the glenosphere 28.

It should also be pointed out that in each of the accompanying FIGS. 5 to 10 and 12, 13 the reverse joint surface of the glenosphere is indicated by reference numeral 33.

All the features disclosed in the application documents are claimed as being of inventive significance, insofar as they are novel individually or in combination.

The invention relates also to the described surgical technique, insofar as protection for such a method is possible in conjunction with the described system.

### REFERENCE NUMERALS

10 scapula
11 base plate
12 joint surface member (anatomical)
13 anatomical joint surface
14 rear side
15 anchoring pin
16 anchoring pin
17 anchoring pin
18 front side
19 threaded bore
20 bore
21 bore
22 bone screw
23 bone screw
24 locking projection
25 locking projection
26 dovetail guide means (guide groove)
27 dovetail guide means (guide groove)
28 joint surface member (reverse or glenosphere)
29 securing screw
30 guide rail
31 guide rail
32 bore for securing screw
33 reverse joint surface

What is claimed is:

1. Scapular component of a shoulder joint prosthesis, comprising a base plate anchorable to the scapula and, affixable to the base plate, a joint surface member which defines either an anatomical joint surface or a reverse joint surface, wherein on the rear side of the base plate, which faces the scapula in the implanted state, there is arranged at least one anchoring pin extending obliquely with respect to the base plate in approximately the anterolateral direction.

2. Scapular component according to claim 1, wherein on the rear side of the base plate there are provided at least two anchoring pins extending obliquely with respect to the base plate.

3. Scapular component according to claim 2, wherein the anchoring pins extend within a predetermined angle range, especially an angle range of from 30 degrees to 70 degrees relative to the base plate, either in parallel or, at least in part, diverging from one another, but always in approximately the anterolateral direction.

4. Scapular component according to claim 1, wherein that the at least one anchoring pin adjoins the rear side of the base plate approximately centrally.

5. Scapular component according to claim 4, wherein the approximately centrally arranged anchoring pin bounds a threaded bore which is accessible from the front side of the base plate, to which the joint surface member is attachable.

6. Scapular component according to claim 1, wherein the primary anchoring of the base plate the latter has at least one bore for receiving a bone screw in such a way that the bone screw extends in the angular region between base plate and anchoring pin, especially in the region of the angle bisecting line between base plate and anchoring pin.

7. Scapular component according to claim 6, wherein the bone screw is held within the associated bore in the base plate either unidirectionally or polydirectionally, preferably at a fixed angle in each case.

8. Scapular component according to claim 6, wherein the case of two or more bone screws the latter extend either in parallel or diverging from one another, the divergence in the latter case being in the range of from 0 degrees to 45 degrees.

9. Scapular component according to claim 1, wherein the joint surface member is pushable and/or lockable onto the front side of the base plate and is thus also exchangeable.

10. Scapular component according to claim 9, wherein the joint surface member is fixable on the base plate by means of a securing screw passing through the joint surface.

11. Scapular component according to claim 10, wherein the securing screw is screwable into the threaded bore which is formed in the approximately centrally arranged anchoring pin.

12. Scapular component according to claim 9, wherein the front side of the base plate there are formed at least one guide grooves or guide ribs extending approximately anteroposteriorly in the implanted state, which grooves or ribs correspond to complementary guide elements on the underside of the respective associated joint surface member.

13. Scapular component according to claim 9, wherein the front side of the base plate has at least one locking projection which is arranged especially at the edge and corresponds to a complementary locking opening in the joint surface member or vice versa.

14. Scapular component according to claim 1, wherein the joint surface member, especially in the case of reverse construction thereof, is anchorable on the base plate either centrally or eccentrically relative to the geometric centre axis thereof.

15. Scapular component according to claim 8, wherein the case of two or more bone screws the latter extend either in parallel or diverging from one another, the divergence in the latter case being in the range of from 15 degrees to 25 degrees.

16. Scapular component of a shoulder joint prosthesis, comprising a base plate anchorable to the scapula and, affixable to the base plate, a joint surface member which defines either an anatomical joint surface or a reverse joint surface, wherein on the rear side of the base plate, which faces the scapula in the implanted state, there is arranged at least one anchoring pin extending obliquely with respect to the base plate in approximately the anterolateral direction and wherein the primary anchoring of the base plate the latter has at least one bore for receiving a bone screw in such a way that the bone screw extends in the angular region between base plate and anchoring pin.

17. Scapular component according to claim 16, wherein the primary anchoring of the base plate the latter has at least one bore for receiving a bone screw in such a way that the bone screw extends in the region of the angle bisecting line between base plate and anchoring pin.

18. Scapular component according to claim 16, wherein the bone screw is held within the associated bore in the base plate either unidirectionally or polydirectionally, preferably at a fixed angle in each case.

19. Scapular component according to claim 16, wherein the case of two or more bone screws the latter extend either in parallel or diverging from one another, the divergence in the latter case being in the range of from 0 degrees to 45 degrees.

20. Scapular component according to claim 19, wherein the case of two or more bone screws the latter extend either in parallel or diverging from one another, the divergence in the latter case being in the range of from 15 degrees to 25 degrees.
